(19) United States
(12) Patent Application Publication
(10) Pub. No.: US 2026/0279100 A1
(43) Pub. Date: Sep. 17, 2026

(54) ONLINE LEARNING WITH FACIAL EMOTION ANALYSIS

(71) Applicant: Abu Dhabi University, Abu Dhabi (AE)

(72) Inventors: Murad M. Al-Rajab, Abu Dhabi (AE); Kevin John, Abu Dhabi (AE); Rawan Elabyed, Abu Dhabi (AE); Salma Said, Abu Dhabi (AE); Saeed Alhmoudi, Abu Dhabi (AE)

(73) Assignee: Abu Dhabi University, Abu Dhabi (AE)

(21) Appl. No.: 19/080,855

(22) Filed: Mar. 15, 2025

(51) Int. Cl. G06V 40/16; A61B 5/00; G06V 10/764; G06V 10/82

(52) U.S. Cl. CPC G06V 40/174; G06V 10/764; G06V 10/82; G06V 40/172; A61B 5/4809

(57) ABSTRACT

A device performs facial expression analysis. The device performs emotional analysis for a person who is interacting with a computer via a camera device. The device determines whether the person is showing an indication of sleepiness.

### BACKGROUND

Recently, the widespread adoption of online education has heightened concerns regarding the effectiveness and motivation levels of online students compared to those in traditional face-to-face classes. The sudden shift to online classes due to the COVID-19 pandemic has impacted almost 95% of the world's student population, marking one of the most abrupt disruptions in the history of education. According to UNESCO, nearly half of the world's students were still affected by partial or complete school closures a year after the COVID-19 outbreak began. Additionally, approximately 1.4 billion students in 186 countries were affected by school closures due to the pandemic.

The lack of face-to-face interaction has made it difficult to accurately determine who is present. This process is repetitive, time-consuming, and prone to human error. For instance, it was common for several students to sign-in on behalf of absent peers or to falsely report their presence. On the other hand, a student's face is a critical element for conveying emotion and is key to the teaching and learning experience. Facial expressions offer insights into a student's emotional state, which is crucial for teachers to understand. Unfortunately, the transition to online learning platforms in response to the COVID-19 pandemic has significantly restricted this vital mode of communication. There is currently no system or method that resolves the above issues.

### BRIEF DESCRIPTION OF DRAWINGS

FIG. 1 is a diagram of an example online learning system;

FIG. 2 is a diagram of an example server architecture;

FIG. 3 is a diagram of an example learning system;

FIG. 4 is a diagram of an example table;

FIG. 5 is a diagram of an example flow process;

FIG. 6 is a diagram of an example table;

FIG. 7 is a diagram of an example graph;

FIG. 8 is a diagram of an example image;

FIG. 9 is a diagram of example images;

FIGS. 10A, 10B, and 10C are diagrams of example graphs;

FIG. 11 is a diagram of example images;

FIG. 12 is a diagram of an example flow process;

FIG. 13 is a networking environment; and

FIG. 14 is a diagram of a computer.

### DETAILED DESCRIPTION OF PREFERRED EMBODIMENTS

The following detailed description refers to the accompanying drawings. The same reference numbers in different drawings may identify the same or similar elements.

Systems, devices, and/or methods described herein develop an online learning system that is an AI-powered Assistant Mentor platform, which assists with online learning. In embodiments, the systems, devices, and/or methods described herein perform facial expression and emotional analysis for students attending online classes. In embodiments, this includes providing real-time feedback and instructor alerts, enabling the instructor to gain insights into students' emotions, signs of sleepiness, and assist them in taking appropriate actions to enhance their learning experience. In embodiments, the online learning system simplifies attendance management and support grade prediction to assist struggling students and enhance their performance.

In embodiments, the described online learning system enhances the online educational experience by employing AI (artificial technologies) technologies and algorithms. In embodiments, the online learning system addresses the challenges associated with real-time emotional monitoring. In embodiments, the primary goal is to measure student engagement during lectures by analyzing emotions, indicative of engagement levels, using a facial recognition framework. In embodiments, this framework includes an automated attendance system, assesses student alertness, and predicts grades. Accordingly, the online learning system uses advanced image detection and Deep Learning (DL) techniques for facial recognition to assess student engagement through emotion analysis and attendance recording. In embodiments, the online learning system uses Machine Learning (ML) algorithms to predict student assessment performance. In embodiments, the user interface platform is customized based on the user, whether an instructor or student.

In embodiments, the online learning system automates the attendance process and introduces an AI-based monitoring system that uses advanced DL facial recognition algorithms to track real-time student emotions, speech, and attendance. In embodiments, the online learning system will provide instructors with an instant performance dashboard, helping them understand student emotions and engagement levels during online classes more effectively. Additionally, the online learning system predicts future student performance by analyzing their ongoing assessment results, moving beyond traditional evaluations that only consider past achievements.

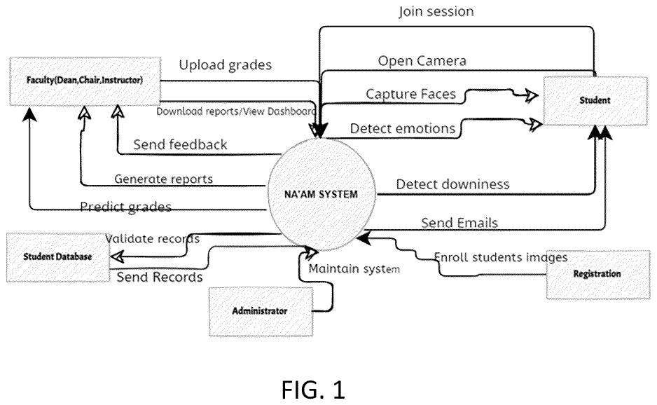

To enhance educational quality and accessibility at minimal cost, particularly for students who cannot attend physical classrooms, such as those with special needs. This approach aligns with sustainable development goals by improving education accessibility and effectiveness. FIG. 1 describes an overview diagram of how the online learning system. As shown in FIG. 1, the online learning system uses detected information about a student's face during class along with grades and other information to further determine whether a student is effectively learning in the online environment.

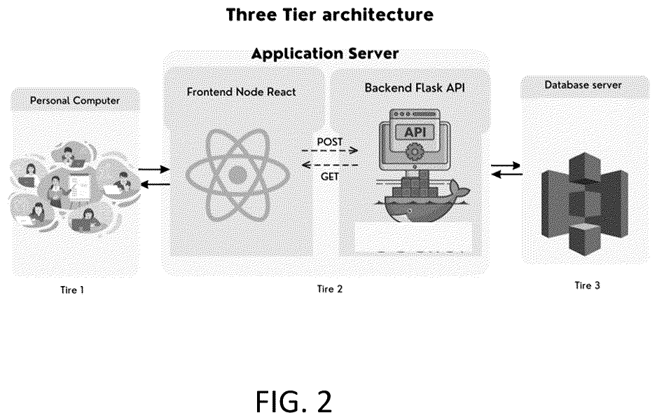

FIG. 2 describes a three-tier architecture for the online learning system. As shown in FIG. 2, the architecture employs a client-server model that segregates presentation, business logic, and data access layers. In embodiments, the presentation layer oversees the user interface and interactions, the business logic layer manages application logic and data validation, and the data access layer handles database queries and updates. In embodiments, the primary purpose of the described architecture is to organize application components into distinct, logical layers, thereby enhancing the maintainability and scalability of the application for future updates.

In embodiments, to develop user interfaces that facilitate interaction with our system, the user interface development framework may be written in JavaScript. For the server side of the online learning system, Node JS, Docker and Flask can be used for deployment. In embodiments, Docker container technology may be used to manage dependency issues, opting for Docker Engine Desktop. Flask, a Python-based micro web application framework. In embodiments, an API (Application Programming Interface) in Flask to handle user login functionality. This API function, written in Python, communicates with the React application via the POST method. It serves as an intermediary, allowing the Python code to interact with the front end without direct contact. For this communication, REST API may be used. Furthermore, a database server is also provided.

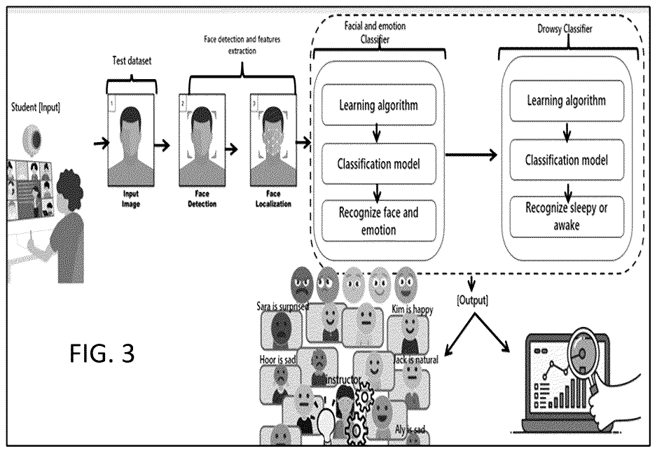

FIG. 3 is an example overview of the online learning system. As shown in FIG. 3, the online learning system starts with the student input, capturing live video/images through webcams or devices for facial recognition and emotion analysis. Face detection identifies faces using deep learning techniques (e.g., such as a CNN detector), followed by face localization that detects facial landmarks (eyes, nose, mouth) for precise feature extraction. In embodiments, the facial and emotion classifier uses a pre-trained ResNet model for facial encoding, categorizes emotions (anger, neutral, disgust, fear, happiness, sadness, surprise), and maps detected emotions to students in real-time. Accordingly, the drowsy classifier, trained on alertness and drowsiness datasets, detects sleepiness through facial cues like eye closure and head nodding, recognizing whether a student is sleepy or awake. The output visualization displays student emotions via emojis and names, provides an instructor dashboard showing engagement, drowsiness, and emotion trends, and generates reports with performance metrics.

In embodiments, the online learning system improves upon the traditional manual attendance system and enhance real-time emotional monitoring during e-learning. In embodiments, advanced image detection is used. In embodiments, students'engagement by monitoring their emotions using Dlib facial recognition technology. After evaluating various models, a pre-trained “Resnet” model is chosen from Dlib for facial encoding in our pipeline.

In embodiments, during the E-learning process, students attend classes via devices equipped with cameras, such as tablets, laptops, or PCs with webcams. In embodiments, the online learning system uses these cameras to capture real-time data on students' presence and emotions, which will help assess class sentiment and attendance. In embodiments, the online learning system can also integrate with CCTV in physical classrooms to enhance the educational process. In embodiments, the electronic imagery received by the online learning system can extract specific facial features for identity verification. In embodiments, only the relevant instructor will access the images, similar to a lockdown browser's privacy approach.

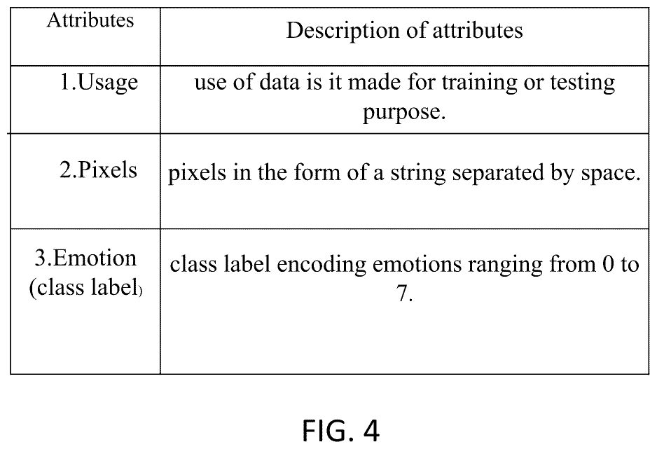

To train the online learning system, a facial expression dataset can be used. In embodiments, this dataset includes approximately 35,888 grey-scale images, each 48×48 pixels, categorized into seven emotions: anger, neutral, disgust, fear, happiness, sadness, and surprise. Each image is labeled with the corresponding emotion. FIG. 4 describes a table that outlines the attributes of the images: pixels, usage, and index. In embodiments, the “pixels” attribute is an integer representing the grayscale intensity of each pixel in the 48×48 image. In embodiments, the “usage” attribute indicates whether the image is designated for training, public testing, or private testing. In embodiments, the “index” attribute provides the emotion index (0=Angry, 1=Disgust, 2=Fear, 3=Happy, 4=Sad, 5=Surprise, 6=Neutral).

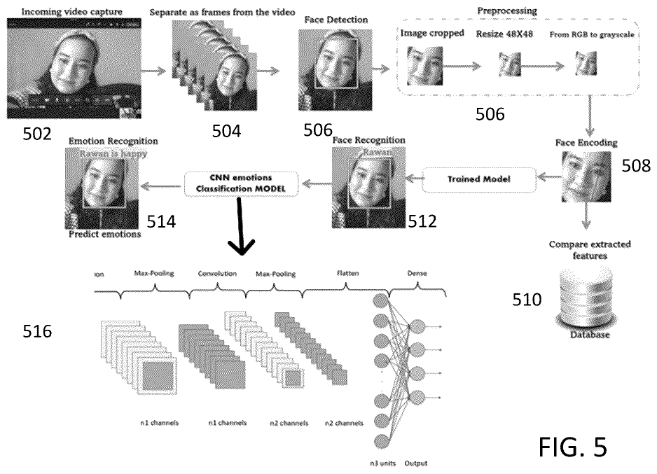

FIG. 5 is an example process that describes how a model is trained. In embodiments, a set of preprocessing techniques must be applied to ensure the model is trained on a high-quality dataset. At step 502, electronic video content is received. At step 504, the electronic video content is parses into multiple frames. At step 506, image preprocessing & augmentation, for the dataset is conducted. In embodiments, image augmentation in ML model is used to enhance the accuracy and precision of models used by the online learning system.

In embodiments, the image augmentation is beneficial when working with low-quality datasets or images that are challenging to process using standard computer vision techniques. In embodiments, image augmentation is necessary to achieve acceptable accuracy levels for specific tasks or datasets. For example, setting rescale=1./255 normalizes pixel values to a range between 0 and 1, converting them from the original range of 0 to 255. In embodiments, image augmentation also allows for image rotation between 0 and 90 degrees. Additionally, the image augmentation can be used to correct images that are not well-represented, filling in missing data. At step 508, the feature extraction is performed on the preprocessed images to identify key facial landmarks and characteristics essential for face recognition and emotion classification.

In embodiments, the extracted features are then encoded to create a compact representation of the face, which is compared against a database of known faces for identification. Accordingly, this encoding process converts facial data into numerical values that are used as input for the trained model. The CNN emotions classification model processes these features through convolution, max-pooling, flattening, and dense layers to predict the student's emotion. The output of the model provides the detected emotion (e.g., “Rawan is happy”) and confirms the recognized face identity, which is then stored and visualized for real-time feedback and analysis.

At step 510, data splitting occurs where a dataset is divided into sets for both model training and validation; the training phase uses 70% of the dataset, while 30% of the dataset is used for model validation. Additionally, pixel values were converted from strings to integers, and emotion labels were converted from integers to categorical types. At step 512, image classification occurs where datasets for our ML models are used to classify. In embodiments, a single black and white image is used as a 3D tensor with the dimensions [width, height, color channels]. For a set of 1,000 black and white images, a 4D tensor with the dimensions [sample size, width, height, color channels] is used. For example, a set of 1000 black and white images, each 640×480 pixels, are represented as a 4D tensor of shape [1000, 640, 480, 1—Standard shape notation for images] 1000 is number of images, 640 is image width in pixels, 480 is image height in pixels and 1 represents the color channel which is grayscale.

At step 514, the classification process is performed where the extracted features are passed through the trained Convolutional Neural Network (CNN) model to predict the emotion and recognize the face. The CNN model processes the input through convolution layers, max-pooling layers, flattening, and dense layers to output a classification label corresponding to different emotions (e.g., happy, sad, angry, surprised). Simultaneously, the face recognition module compares the encoded features against the database to identify the individual. The final output provides both the recognized face identity and the predicted emotion, which is then used for visualization and stored for further analysis.

At step 516, the online learning system employs a Convolutional Neural Network (CNN) to classify emotions based on facial expressions. The CNN begins with convolution layers that extract spatial features from the input image by applying filters to detect patterns such as edges, textures, and distinct facial characteristics. These features are then passed through max-pooling layers, which reduce the spatial dimensions of the feature maps, minimizing computational complexity while retaining essential information. The output of the pooling layers is flattened into a one-dimensional vector, preparing it for the dense (fully connected) layers.

These dense layers analyze the extracted features and perform classification by mapping them to output classes representing various emotions like happiness, sadness, anger, and surprise. The final output layer provides the predicted emotion category for the detected face. This process enables the system to learn and identify complex patterns in facial expressions, ensuring accurate emotion classification.

In embodiments, the online learning system is trained by progressively refining its ability to make accurate classifications while ensuring learning stability. The model adjusts its learning dynamically, preventing sudden changes that could stop convergence. To measure performance, the online learning system calculates the difference between predicted and actual values, systematically minimizing errors through repetitive updates. Training is conducted in batches to optimize efficiency and repeated over multiple cycles to enhance accuracy. A separate validation dataset is utilized to evaluate the model's generalization capability, ensuring it learns meaningful patterns rather than merely memorizing data. After training, the optimized model is stored for further testing and deployment in real-world applications.

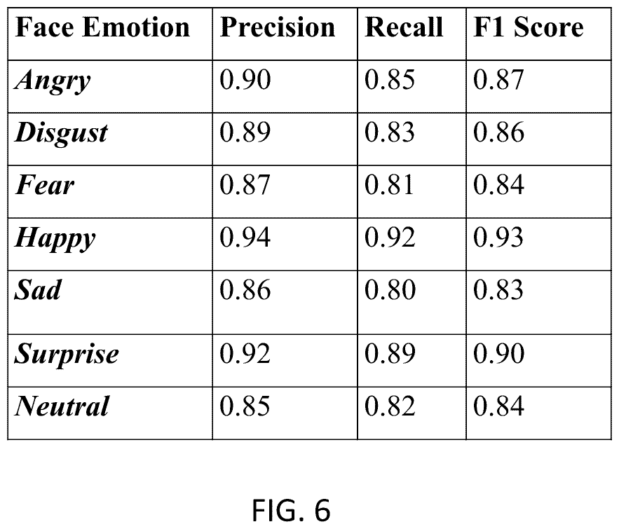

FIG. 6 is a table that describes detailed results obtained from model validation used for each emotion. In embodiments, the Face Emotion Classifier Model shows promising validation results for the seven emotions types considered in this study, with high precision, recall, and F1 scores, particularly for Happy and Surprise.

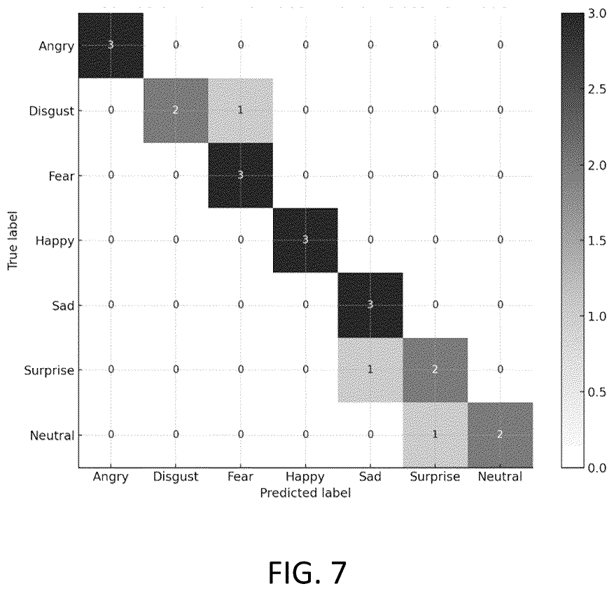

Additionally, the confusion matrix, described in FIG. 7, shows that the model accurately classifies most emotions. The diagonal values represent correct classifications, with emotions such as Angry, Fear, Happy, and Sad being identified with perfect accuracy. However, some misclassifications occur, particularly with Disgust, which is sometimes mistaken for Fear, and Surprise, which is misclassified as Sad or Neutral in certain instances. Additionally, Neutral is occasionally confused with Surprise, highlighting areas where the model struggles to differentiate similar expressions. The overall pattern suggests that while the model performs well.

In embodiments, the online learning system can recognize faces from any image folder with high accuracy. In embodiments, following steps detail how the learning system is developed and trained. First, face imagery is imported into the recognition library, which handles all recognition tasks. In embodiments, the images are loaded into the library and assigned a name. Then, using a face locations method, all faces are identified in the image, which returns a 2D array of bounding boxes around each face detected using the CNN face detector. Furthermore, the model also includes capturing facial images of students directly from a live stream. In embodiments, facial features are then recognized, including the eyes, eyebrows, nose, mouth, and chin for each stored face.

In embodiments, training with known faces and creating and linking encodings is conducted. In embodiments, an array is constructed that links facial encodings with corresponding names, enabling the online learning system to match each encoding to the appropriate identity. After identifying and mapping the facial landmarks present in an image, we proceed to recognize the individual's identity. We trained the model by uploading and encoding images of known individuals, thereby training the model to correctly identify these faces. In embodiments, video frames are processed by resizing and converting the video frames to RGB color to align with the requirements of the recognition library.

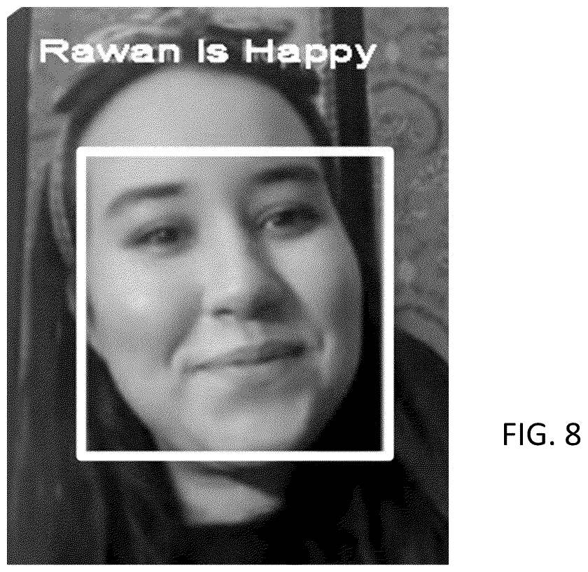

In embodiments, the system compares the encodings from the video stream with pre-existing encodings in the database. If a match is found, it identifies the face based on the encoding that most closely matches. In embodiments, the online learning system displays the results by placing a bounding box around the recognized face and labeling it with the name at the bottom of the box. In embodiments, this systematic approach enables our model to effectively recognize and verify faces in real-time during online classes. In embodiments, the online learning system can detect faces, identify students as shown in FIG. 8 with the image showing the student's name, and showing high efficiency in a real time classroom scenario.

In embodiments, the online learning system also conducted grade prediction by using linear regression. In embodiments, the features of the dataset are composed of homework scores, test scores, and the target label: the total score. In embodiments, the dataset is divided into a training set and a testing set, with the training set comprising two-thirds of the total data and the remaining data allocated to the testing set.

In embodiments, two distinct approaches were utilized to develop the linear regression model. This included a polyfit process to compute the slope and intercept for the best-fit line, and a linear regression to determine the coefficient (R-squared) as a measure of model accuracy. In embodiments, the grade prediction model developed here reveals promising performance, as evidenced by the evaluation metrics. In embodiments, the model achieved an R-squared value of 0.85, indicating a good level of accuracy based on the input features. The Mean Absolute Error (MAE) stands at 4.5, highlighting the model's precision in predicting student grades with minimal average error.

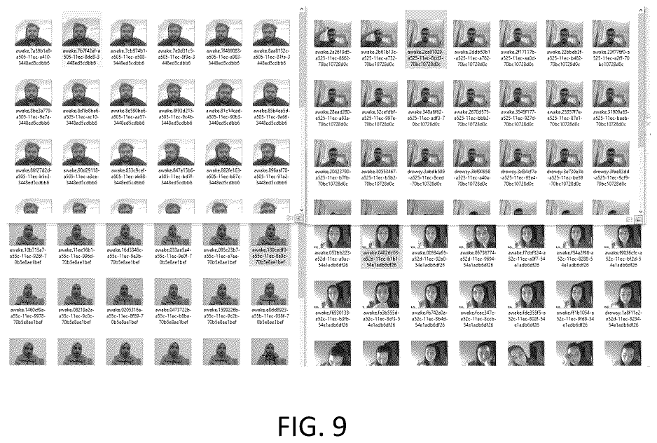

In embodiments, a custom developed dataset that contains a particular number of hours or RGB video footage for a particular number of students. For example, approximately 30 hours of RGB video footage would be provided for 60 students. From each participant, 180 short videos may be collected and categorized into three states: alertness, low vigilance, and drowsiness. In embodiments, feature points from 44 videos of 22 individuals are extracted using this dataset. Furthermore, gathered comprehensive data for both alert and drowsy states is obtained. To obtain images of the students, one frame per second is extracted. Additionally, the model is enhanced by developing a dataset. For each execution of the script, we extracted 20 frames labeled as ‘drowsy’ are extracted and another 20 frames labeled as ‘awake,’are extracted as shown in FIG. 9.

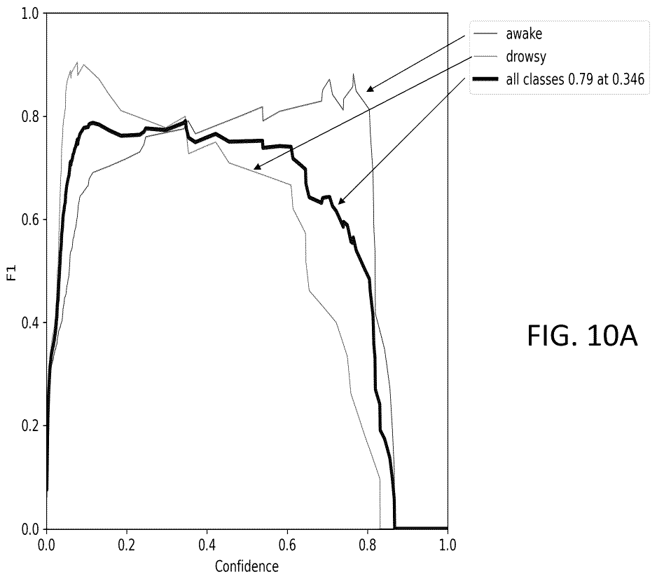

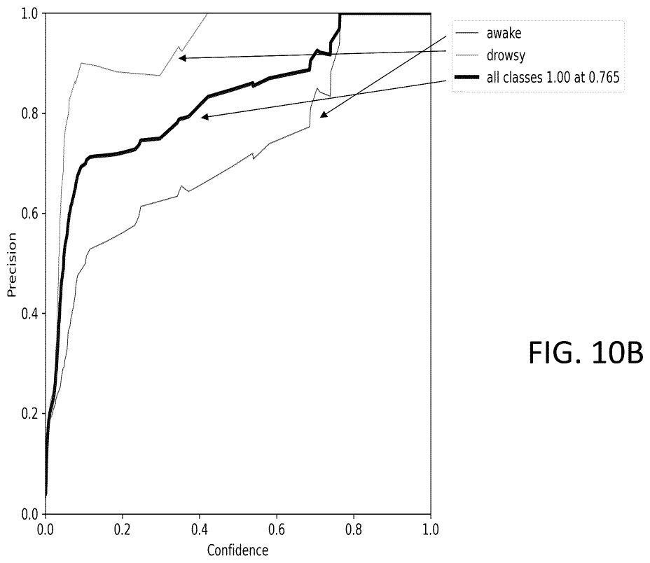

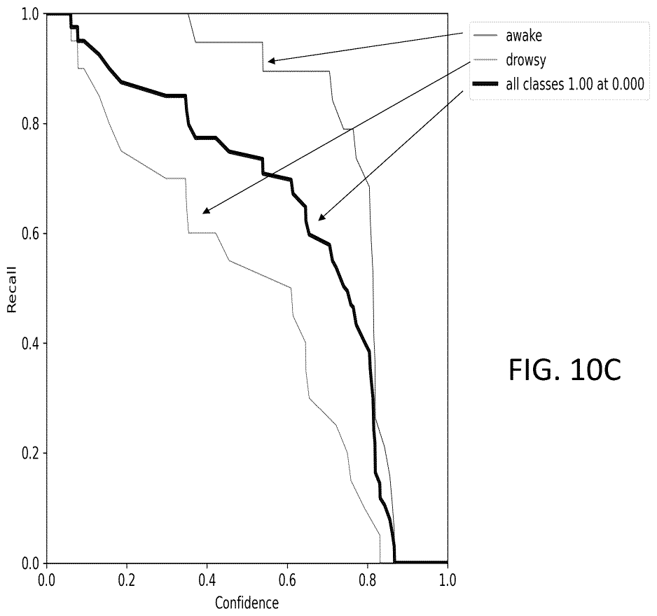

In embodiments, additional model attributes are determined, including hyperparameters, number of classes, and class weights. Training then commences with the setting of epochs and iterations. Additionally, mapping and various other configurations related to training the drowsiness detection algorithm model are established. Various metrics have also been applied to more accurately verify the results of the drowsiness detection algorithm. FIG. 10A is an example graph that displays the F1 score obtained for our model. As illustrated, F1 score for drowsiness gradually decreases as confidence increases, while the awake F1 score initially rises with increasing confidence but sharply drops back to 0 at the 0.89 mark on the confidence axis.

Another metric used is the precision curve is shown in FIG. 10B. With increasing confidence, all three lines representing precision also rise. Additionally, the recall metric, represented by the recall curve is shown in FIG. 10C, which gradually decreases as confidence increases.

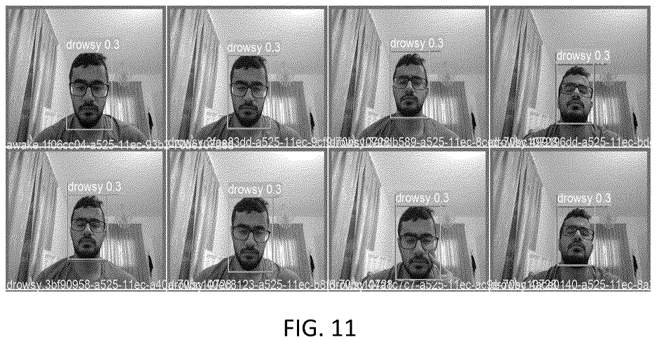

Lastly, the drowsiness detection algorithm is demonstrated in FIG. 11. In embodiments, the algorithm successfully detects the face of a student and verifies that (s)/he is indeed in a drowsy state, according to the model we have implemented.

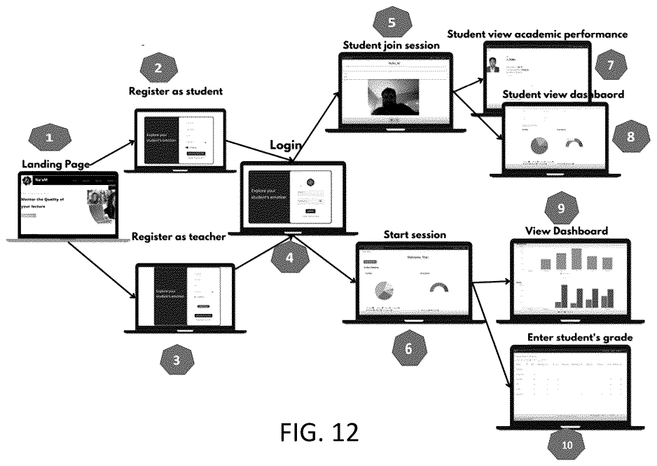

FIG. 12 describes an example process of the described online learning system. As shown in FIG. 12, users begin by navigating to the landing page which directs them to the registration page. Here, they have the option to login if they already have an account or to create a new account if they do not. Users can register as either an instructor or a student.

First, the instructor and the student go through the sign-up process using the screen shown in FIG. 11, as shown in screen 1. Then, they register as a student or an instructor, as shown in screen 2 or screen 3, respectively. Once registered, the student or the instructor can login, as shown in screen 4.

Upon successful login, the student is directed to a page listing available sessions. The system will first request permission to activate the camera. This enables the system to capture the student's face, emotions, level of alertness, and attendance. Students can then access their profiles to review their academic performance. The “Join Session” button is prominently displayed, as illustrated in FIG. 11. Once clicked, the system begins capturing the required information based on each operational model, also detailed in screens 5, 7, and 8 in FIG. 12.

On the other hand, instructors, upon logging into the system, can create a session for the class. This session is then made available to students to join. Additionally, instructors can terminate the session at any time. Following the session, they have the capability to assess and record comprehensive details about each student's emotions, alertness, attendance, and grade prediction, as illustrated in screens 6, 9, and 10 of FIG. 11.

FIG. 11 is discussed further. Additionally, instructors can review the collective emotional state of the students for each session. By clicking on a session name, they can access a list of students who attended, as shown in screen 9, observe levels of drowsiness, and analyze overall student sentiment through various graphical representations, as shown in screen 6. For example, emotions are displayed in a pie chart, and hovering over the chart reveals the percentage breakdown of current sentiments. As shown on the left side of the screen 6, emoji icons are incorporated to enhance the visual appeal and clarity of the data presented. The detection of drowsiness is depicted through an indicator that shows whether the class is alert or experiencing drowsiness, as illustrated on right side of the screen 6.

For the attendance taking and grade prediction, a Bar chart is suitable for this representation part, as shown in screen 9. Once the instructor enters the grades of the different assessments of students, the system will predict the total score of all students and displays it as the last column in the table as shown in Screen 10. Additionally, instructors have the ability to add students, filter them based on their grades, and export the page in various formats such as CSV or Excel for further analysis. Moreover, if the system predicts a student's grade to be below 50%, the instructor will see a button for sending an automated email. Upon clicking this button, an email is sent to the affected students, advising them to seek tutoring assistance.

Our proposed system is a comprehensive solution that can be used in several settings such as educational institutions and in companies to track the attendance and emotions of employees. Therefore, our system addresses the limitations of the above-mentioned applications by integrating promising accuracy across different lighting conditions and skin tones, comprehensive features including drowsiness detection, data privacy. This makes our system a better solution for face detection, emotion recognition, and drowsiness detection, suitable for large-scale applications

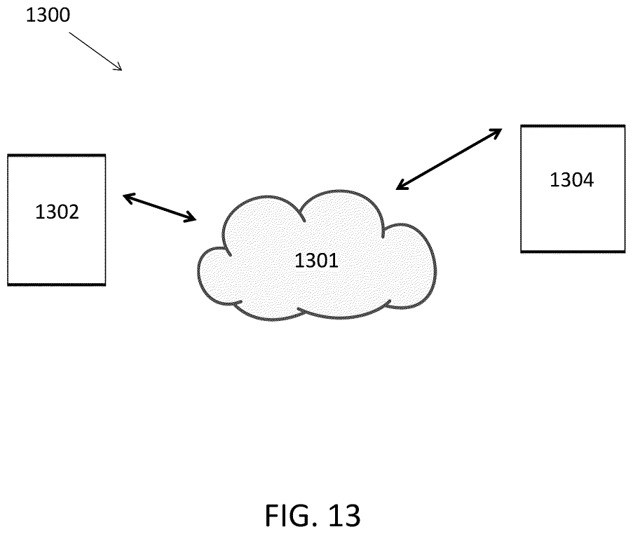

FIG. 13 is a diagram of example environment 1300 in which systems, devices, and/or methods described herein may be implemented. FIG. 13 shows network 1301, user device 1302, and server 1304.

Network 1301 may include a local area network (LAN), wide area network (WAN), a metropolitan network (MAN), a telephone network (e.g., the Public Switched Telephone Network (PSTN)), a Wireless Local Area Networking (WLAN), a WiFi, a hotspot, a Light fidelity (LiFi), a Worldwide Interoperability for Microware Access (WiMax), an ad hoc network, an intranet, the Internet, a satellite network, a GPS network, a fiber optic-based network, and/or combination of these or other types of networks. Additionally, or alternatively, network 1301 may include a cellular network, a public land mobile network (PLMN), a second generation (2G) network, a third generation (3G) network, a fourth generation (4G) network, a fifth generation (5G) network, and/or another network.

In embodiments, network 1301 may allow for devices describe any of the described figures to electronically communicate (e.g., using emails, electronic signals, URL links, web links, electronic bits, fiber optic signals, wireless signals, wired signals, etc.) with each other so as to send and receive various types of electronic communications.

User device 1302 may include any computation or communications device that is capable of communicating with a network (e.g., network 1301). For example, user device 1302 may include a radiotelephone, a personal communications system (PCS) terminal (e.g., that may combine a cellular radiotelephone with data processing and data communications capabilities), a personal digital assistant (PDA) (e.g., that can include a radiotelephone, a pager, Internet/intranet access, etc.), a smart phone, a desktop computer, a laptop computer, a tablet computer, a camera, a personal gaming system, a television, a set top box, a digital video recorder (DVR), a digital audio recorder (DUR), a digital watch, a digital glass, or another type of computation or communications device.

User device 1302 may receive and/or display content. The content may include objects, data, images, audio, video, text, files, and/or links to files accessible via one or more networks. Content may include a media stream, which may refer to a stream of content that includes video content (e.g., a video stream), audio content (e.g., an audio stream), and/or textual content (e.g., a textual stream). In embodiments, an electronic application may use an electronic graphical user interface to display content and/or information via user device 1302. User device 1302 may have a touch screen and/or a keyboard that allows a user to electronically interact with an electronic application. In embodiments, a user may swipe, press, or touch user device 1302 in such a manner that one or more electronic actions will be initiated by user device 1302 via an electronic application.

User device 1302 may include a variety of applications, such as, for example, an e-mail application, a telephone application, a camera application, a video application, a multi-media application, a music player application, a visual voice mail application, a contacts application, a data organizer application, a calendar application, an instant messaging application, a texting application, a web browsing application, a blogging application, and/or other types of applications (e.g., a word processing application, a spreadsheet application, etc.). In embodiments, user device 1302 may be used to generate graphs and images (such as those described in the above figures). In embodiments, user device 1302 may communicate with server 1304. In embodiments, server 1304 may include any computation or communications device that is capable of communicating with a network; and can store electronic information and conduct one or more steps discussed in the above figures to determine a student's physical state.

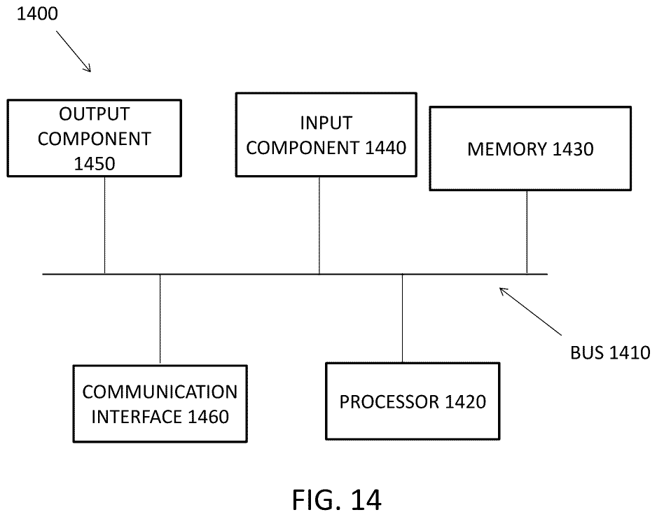

FIG. 14 is a diagram of example components of a device 1400. Device 1400 may correspond to user device 1402, or user device 1404. Alternatively, or additionally, user device 1302 and server 1304 may include one or more devices 1400 and/or one or more components of device 1400.

As shown in FIG. 14, device 1400 may include a bus 1410, a processor 1420, a memory 1430, an input component 1440, an output component 1450, and a communications interface 1460. In other implementations, device 1400 may contain fewer components, additional components, different components, or differently arranged components than depicted in FIG. 14. Additionally, or alternatively, one or more components of device 1400 may perform one or more tasks described as being performed by one or more other components of device 1400.

Bus 1410 may include a path that permits communications among the components of device 1400. Processor 1420 may include one or more processors, microprocessors, or processing logic (e.g., a field programmable gate array (FPGA) or an application specific integrated circuit (ASIC)) that interprets and executes instructions. Memory 1430 may include any type of dynamic storage device that stores information and instructions, for execution by processor 1420, and/or any type of non-volatile storage device that stores information for use by processor 1420. Input component 1440 may include a mechanism that permits a user to input information to device 1400, such as a keyboard, a keypad, a button, a switch, voice command, etc. Output component 1450 may include a mechanism that outputs information to the user, such as a display, a speaker, one or more light emitting diodes (LEDs), etc.

Communications interface 1460 may include any transceiver-like mechanism that enables device 1400 to communicate with other devices and/or systems. For example, communications interface 1460 may include an Ethernet interface, an optical interface, a coaxial interface, a wireless interface, or the like.

In another implementation, communications interface 1460 may include, for example, a transmitter that may convert baseband signals from processor 1420 to radio frequency (RF) signals and/or a receiver that may convert RF signals to baseband signals. Alternatively, communications interface 1460 may include a transceiver to perform functions of both a transmitter and a receiver of wireless communications (e.g., radio frequency, infrared, visual optics, etc.), wired communications (e.g., conductive wire, twisted pair cable, coaxial cable, transmission line, fiber optic cable, waveguide, etc.), or a combination of wireless and wired communications.

Communications interface 1460 may connect to an antenna assembly (not shown in FIG. 14) for transmission and/or reception of the RF signals. The antenna assembly may include one or more antennas to transmit and/or receive RF signals over the air. The antenna assembly may, for example, receive RF signals from communications interface 1460 and transmit the RF signals over the air, and receive RF signals over the air and provide the RF signals to communications interface 1460. In one implementation, for example, communications interface 1460 may communicate with network 1301.

As will be described in detail below, device 1400 may perform certain operations. Device 3000 may perform these operations in response to processor 1420 executing software instructions (e.g., computer program(s)) contained in a computer-readable medium, such as memory 1430, a secondary storage device (e.g., hard disk, CD-ROM, etc.), or other forms of RAM or ROM. A computer-readable medium may be defined as a non-transitory memory device. A memory device may include space within a single physical memory device or spread across multiple physical memory devices. The software instructions may be read into memory 1430 from another computer-readable medium or from another device. The software instructions contained in memory 1430 may cause processor 1420 to perform processes described herein. Alternatively, hardwired circuitry may be used in place of or in combination with software instructions to implement processes described herein. Thus, implementations described herein are not limited to any specific combination of hardware circuitry and software.

It will be apparent that example aspects, as described above, may be implemented in many different forms of software, firmware, and hardware in the implementations illustrated in the figures. The actual software code or specialized control hardware used to implement these aspects should not be construed as limiting. Thus, the operation and behavior of the aspects were described without reference to the specific software code—it being understood that software and control hardware could be designed to implement the aspects based on the description herein.

Even though particular combinations of features are recited in the claims and/or disclosed in the specification, these combinations are not intended to limit the disclosure of the possible implementations. In fact, many of these features may be combined in ways not specifically recited in the claims and/or disclosed in the specification. Although each dependent claim listed below may directly depend on only one other claim, the disclosure of the possible implementations includes each dependent claim in combination with every other claim in the claim set.

While various actions are described as selecting, displaying, transferring, sending, receiving, generating, notifying, and storing, it will be understood that these example actions are occurring within an electronic computing and/or electronic networking environment and may require one or more computing devices, as described in FIG. 29, to complete such actions.

No element, act, or instruction used in the present application should be construed as critical or essential unless explicitly described as such. Also, as used herein, the article “a” is intended to include one or more items and may be used interchangeably with “one or more.” Where only one item is intended, the term “one” or similar language is used. Further, the phrase “based on” is intended to mean “based, at least in part, on” unless explicitly stated otherwise.

In the preceding specification, various preferred embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

What is claimed is:

1. A device, comprising:
one or more processors to:
perform facial expression analysis;
perform emotional analysis for a person who is interacting with a computer via a camera device; and
determine whether the person is showing an indication of sleepiness.

2. The device of claim 1, wherein the performing the facial expression analysis includes using advanced image detection and Deep Learning (DL) techniques, and wherein the one or more processors are further to:
use Machine Learning (ML) algorithms to predict student assessment performance.

3. The device of claim 1, wherein the facial expression analysis includes encodings from a video stream with pre-existing encodings in a database.

4. The device of claim 1, wherein the indication of sleepiness generates an electronic indicator displayed on an electronic screen.

5. The device of claim 1, wherein the device includes:
a presentation layer oversees the user interface and interactions,
a business logic layer manages application logic and data validation, and
a data access layer handles database queries and updates.

6. A method, comprising:
performing, by a computing device, facial expression analysis;
performing, by the computing device, emotional analysis for a person who is interacting with a computer via a camera device; and
determining, by the computing device, whether the person is showing an indication of sleepiness.

7. The method of claim 6, wherein the performing the facial expression analysis includes using advanced image detection and Deep Learning (DL) techniques, and wherein the method further comprising:
using Machine Learning (ML) algorithms to predict student assessment performance.

8. The method of claim 6, wherein, the computing device includes:
a presentation layer oversees the user interface and interactions,
a business logic layer manages application logic and data validation, and
a data access layer handles database queries and updates.
